(19) United States
(12) Patent Application Publication
(10) Pub. No.: US 2026/0089901 A1
(43) Pub. Date: Mar. 26, 2026

(54) METHOD FOR FABRICATING A CIRCUIT BOARD ARRANGEMENT, CIRCUIT BOARD PRE-ASSEMBLY AND IMPLANTABLE MEDICAL DEVICE COMPRISING A CIRCUIT BOARD ARRANGEMENT

(71) Applicant: BIOTRONIK SE & Co. KG, Berlin (DE)

(72) Inventors: Bjoern BRUNNER, Portland, OR (US); Kenneth M. KRAMER, Lake Oswego, OR (US); Garrett COGBURN, Tigard, OR (US)

(73) Assignee: BIOTRONIK SE & Co. KG, Berlin (DE)

(21) Appl. No.: 19/112,323

(22) PCT Filed: Sep. 19, 2023
(86) PCT No.: PCT/EP2023/075723

§ 371 Date: Mar. 17, 2025

Related U.S. Application Data
(60) Provisional application No. 63/411,880, filed on Sep. 30, 2022.

(30) Foreign Application Priority Data
Oct. 27, 2022 (EP) 22204058.6

(51) Int. Cl. H05K 9/00; A61N 1/39

(52) U.S. Cl. CPC H05K 9/0022; A61N 1/3956

(57) ABSTRACT

A method for fabricating a circuit board arrangement includes: providing a circuit board panel forming at least one circuit board section, a remaining panel section and at least one support portion connecting the at least one circuit board section to the remaining panel section; placing at least one functional component on the at least one circuit board section; and separating the at least one circuit board section from the remaining panel section at the at least one support portion using an excise device, wherein a portion of the at least one functional component extends beyond an edge of the at least one circuit board section, and at least one shield element is arranged on the circuit board panel such a shield element of the at least one shield element is at least partly arranged between the at least one functional component and the edge of the circuit board section.

### CROSS-REFERENCE TO RELATED APPLICATIONS

This application is the United States National Phase under 35 U.S.C. § 371 of PCT International Patent Application No. PCT/EP2023/075723, filed on Sep. 19, 2023, which claims the benefit of European Patent Application No. 22204058.6, filed on Oct. 27, 2022, and U.S. Provisional Patent Application No. 63/411,880 , filed Sep. 30, 2022, the disclosures of which are hereby incorporated by reference herein in their entireties.

### TECHNICAL FIELD

The instant invention relates to a method for fabricating a circuit board arrangement, a circuit board pre-assembly and an implantable medical device comprising a circuit board arrangement.

### BACKGROUND

Generally, a circuit board arrangement comprises one or multiple circuit board sections, which carry electrical or electronic components, for example of an implantable medical device, such as a stimulation device in the shape of a pacemaker device, a defibrillation device or a neuro-stimulation device. Generally herein, a circuit board arrangement is to be enclosed in a housing of the corresponding device, wherein the circuit board sections may be aligned with respect to one another, for example folded or stacked, such that the circuit board sections assume a defined position with respect to one another. In the context of an implantable medical device, for example communication equipment, such as a communication coil, may be placed on a circuit board section of the circuit board arrangement.

An implantable medical device generally shall be configured for implantation into a patient, e.g., into the heart of a patient. An implantable medical device in this context may for example be a pacemaker device, such as a leadless pacemaker, for providing for a pacing action in a patient's heart, or a defibrillator device, such as an implantable cardioverter-defibrillator (ICD), for providing for a defibrillation, or a monitoring device having a sensing function for monitoring for example a cardiac activity of a patient. Implantable medical devices, in particular implantable medical devices which shall be directly implanted into the patient's heart, such as a leadless pacemaker, are small in size, relevant components of the device-such as an energy storage, a control circuitry and a communication unit-being encompassed in the housing in an encapsulated, fluid-tight fashion.

A circuit board arrangement to be fitted in a housing of an implantable medical device, such as a leadless pacemaker device, consequently, is small in size. Within such a circuit board arrangement, for example multiple circuit board sections may be stacked such that an arrangement of electric and electronic components on multiple levels is formed. Herein, to achieve an efficient processing, circuit board sections of a circuit board arrangement are typically manufactured from a larger circuit board panel. During the manufacturing, components are placed on the circuit board panel, upon which the components are joined to the circuit board panel using a soldering process, such as a reflow soldering technique. This allows one to simultaneously and efficiently manufacture circuit board sections of a circuit board arrangement but makes it necessary to separate circuit board sections with components placed thereon from a remaining panel section during the manufacturing.

For separating the circuit board sections, an excise device such as a laser device is used to excise the circuit board panel. In certain circuit board arrangements, components may be used which are large in comparison to a circuit board section and may reach across an edge of the circuit board section carrying the component. As a laser may damage a component if it acts onto the component during the separation step, pre-rout openings are formed within the circuit board panel prior to placing the components on the circuit board panel, such that the circuit board section is partially free from the remaining panel section or another circuit board section. As any pre-rout opening weakens the material of the circuit board panel, pre-rout openings may make processing of the circuit board panel difficult, for example during a solder print step, as the circuit board panel may flex away during the processing. For this reason, at certain support sections a circuit board section is connected with a remaining panel section to support the circuit board section during processing. Consequently, at these support sections the circuit board panel needs to be excised when separating the circuit board section from the remaining panel section.

U.S. Pat. No. 6,245,092 B1 discloses a highly integrated electronic circuit with a flexible substrate which has a component side and an insulating bottom side as well as an arrangement of active and passive electronic components. The components are mounted on bond pads of the substrate and are connected by conductors for establishing a circuit function.

U.S. Pat. No. 6,924,437 B1 discloses a circuit board assembly including a circuit board which defines circuit board pads, a set of surface mount electronic components soldered to a first set of the circuit board pads using a surface mount soldering process, and a set of surface mount coupling devices soldered to a second set of the circuit board pads using the surface mount soldering process.

It is an object of the present invention to provide a method for fabricating a circuit board arrangement, a circuit board pre-assembly usable for fabricating a circuit board arrangement and an implantable medical device comprising a circuit board arrangement which allow for an easy and reliable processing of a circuit board panel, with a reduced risk for damaging components during a separating step for separating a circuit board section from a remaining panel section.

### SUMMARY

In one aspect, a method for fabricating a circuit board arrangement comprises: providing a circuit board panel forming at least one circuit board section, a remaining panel section, and at least one support portion connecting the at least one circuit board section to the remaining panel section; placing at least one functional component on the at least one circuit board section; and separating the at least one circuit board section from the remaining panel section at the at least one support portion using an excise device.

Within the method, one or multiple circuit board sections are formed from a circuit board panel which, in an initial stage, connects the circuit board sections. Using a circuit board panel of this kind allows one to place electric and electronic components on a multiplicity of circuit board sections using, for example, an automated placement machine, allowing one to place components in an automatic fashion at high speed, wherein the circuit board panel with the components placed thereon may subsequently be processed, for example to simultaneously join the components to the circuit board panel, for example using a soldering technique such as a reflow soldering technique.

At the outset of the method, a circuit board section is connected to a remaining panel section by means of one or multiple support portions, such that the circuit board section is supported with respect to the remaining panel section. During the fabrication method, one or multiple components may be placed on the circuit board section, upon which the circuit board section is separated from the remaining panel section at the support portion by cutting the support portion using an excise device, such as a laser device.

At the outset the circuit board panel may extend flatly along a plane of extension. Circuit board sections of the circuit board panel hence are aligned with respect to one another along the plane of extension, wherein, during the fabrication, functional components may be placed e.g. on one side of the circuit board panel in order to provide an electric or electronic function on the circuit board panel.

As a functional component placed on the circuit board section may be comparatively large in size and may reach across an edge of the circuit board section, it may be the case that, in the separating step, there is a risk that an excise device comes into contact with the component, bearing the risk that the component is damaged during the separating step. For this reason it herein is proposed to use a shield element which is placed, prior to the separating step, on the circuit board panel. This shield element protects a functional component from an excise device, hence reducing a risk that the excise device during the separating step may act on the functional component causing damage. Accordingly, it is particularly envisioned according to the invention that a portion of the at least one functional component extends beyond an edge of the at least one circuit board section, on which at least one functional component is arranged, and at least one shield element is arranged on the circuit board panel such that a shield section of the at least one shield element is at least partly arranged between the at least one functional component and the edge of the circuit board section on which the at least one functional component is arranged.

The shield element in particular may serve as a sacrificial element which, after the separating step, may be discarded. By forming the shield section, the shield element prevents contact of a functional component with an excise device, in particular a laser device when excising the support portion to separate the circuit board section.

In one embodiment, the shield element is placed on the circuit board panel such that the shield section of the shield element is (at least partially) placed on the support portion and covers an area of the support portion.

Beneficially, in one embodiment, said step of placing the at least one shield element on the circuit board panel is carried out prior to said step of placing the at least one functional component on the at least one circuit board section. The at least one shield element hence may be placed on the circuit board panel such that the shield section comes to lie immediately on or over the support portion, hence shielding a functional component being placed on top of the shield section from an excise device.

In one embodiment, the at least one shield element comprises a fastening section for fastening the at least one shield element to the circuit board panel. Herein, the fastening section may be placed during the step of placing the at least one shield element on the circuit board panel, on the remaining panel section, such that the at least one shield element is fixed to the remaining panel section and, after separating the circuit board section from the remaining panel section, may be discarded together with the remaining panel section. When placing the shield element on the circuit board panel, the fastening section for example may be placed on a solder pad on the remaining panel section, and may subsequently be joined to the remaining panel section in a soldering process, in particular a reflow process. Once the circuit board section is separated from the remaining panel section, the shield element may be removed from the circuit board section together with the remaining panel section.

In one embodiment, the at least one shield element and the at least one functional component are joined to the circuit board panel using a soldering technique, in particular a reflow soldering technique. The at least one shield element and the at least one functional component for this beneficially are fabricated as Surface Mount Devices (SMD) such that Surface Mount Technologies (SMT) may be used for joining the at least one shield element and the at least one functional component to the circuit board panel. Joining using the soldering technique herein is carried out after placing the at least one shield element and the at least one functional component on the circuit board panel. The joining takes place prior to separating the at least one circuit board section from the remaining panel section during the separating step.

In one embodiment, the at least one shield element is placed on the circuit board panel such that the shield section of the at least one shield element is arranged in between the at least one support portion and a portion of the functional component (when viewed along a normal direction perpendicular to the plane of extension of the circuit board panel). When placing the at least one functional component on the at least one circuit board section, hence, a portion of the functional component reaching across or extending beyond the support portion is placed on top of the shield section of the shield element (when viewed along a normal direction perpendicular to the plane of extension of the circuit board panel), the shield section hence shielding the overhanging portion of the functional component from the support portion. If subsequently the support portion is excised using an excise device such as a laser device, contact between the excise device and the overhanging portion of the functional component is prevented by means of the shield section.

An overhanging functional component may for example be a communication coil, a frame, or a feedthrough assembly to be placed on a circuit board section of comparatively small size. Such a feedthrough assembly may include a conductor extending through and joined with an insulator, and a flange or ferrule having an opening in which the insulator is arranged.

In one embodiment, when placing the shield element on the circuit board panel, the shield element is placed on a first side of the circuit board panel. Likewise, the at least one functional component is placed on the first side of the circuit board panel, such that the shield element and the functional component are placed on the same side of the circuit board panel.

In one embodiment, the excise device is used on a second side of the circuit board panel opposite the first side. The excise device, for example a laser excise device, hence is directed towards the circuit board panel on the second side of the circuit board panel to impinge on the support portion in order to cut the support portion and to separate the circuit board panel from the remaining panel section at the support portion. As the shield element on the first side of the circuit board panel shields the functional component, e.g. a laser beam of an excise device in the form of a laser device may not come into contact with the functional component, such that the risk of damaging the functional component during the separating step is substantially reduced.

In one embodiment, the circuit board panel comprises one or multiple pre-rout openings partially separating the at least one circuit board section from the remaining panel section. The pre-rout openings are formed in the circuit board panel prior to placing the at least one shield element and the at least one functional component on the circuit board panel. Hence, the at least one circuit board section is already partially separated from the remaining panel section prior to placing the at least one shield element and the at least one functional component on the circuit board panel during the manufacturing. Hence, during the step of separating, only the support portion needs to be excised in order to separate the circuit board section from the remaining panel section, wherein the shield element provides for a shielding in the region of the support portion in order to prevent a damaging of a functional component in the region of the support portion.

The at least one pre-rout opening may for example have the shape of a slit formed in the circuit board panel, the at least one pre-rout opening separating an associated circuit board section from the remaining panel section along its length.

In one embodiment, the step of placing the at least one functional component on the at least one circuit board section includes placing the at least one functional component on the at least one circuit board section such that a portion of the functional component reaches across or extends beyond the at least one pre-rout opening (when viewed along the plane of extension of the circuit board panel). A pre-rout opening may in particular be formed in such a region of the circuit board section in which a component of comparatively large size is to be placed which reaches across or extends beyond an edge of the circuit board section. In order to avoid having to excise the circuit board section from the remaining panel section in that region, the pre-rout opening is formed.

In one embodiment, the circuit board panel forms at least two circuit board sections and a flexible connection section connecting the at least two circuit board sections to each other. In this embodiment, the step of separating includes separating the at least two circuit board sections and the flexible connection section from the remaining panel section, upon which the circuit board sections may be folded with respect to one another such that e.g. the circuit board sections may be arranged in a stacked fashion with respect to one another such that they extend in parallel to one another. In this way a stack of circuit board sections may be formed to provide the circuit board arrangement.

In one embodiment, a circuit board arrangement may comprise multiple flexible connection sections, each flexible connection section connecting two neighboring circuit board sections with each other. The circuit board arrangement may, in one embodiment, form a zigzag shape (also denoted as “accordion” shape) in that in an operative state a first circuit board section is connected via a first connection section to a second circuit board section, and the second circuit board section is connected via a second connection section to a third circuit board section. By means of further connection sections further circuit board sections may adjoin the third circuit board section, such that multiple circuit board sections by means of flexible connection sections are interconnected which each other and form a zigzag (accordion) shape. One or multiple standoff elements may be arranged in between the circuit board sections of each pair of neighboring circuit board sections, such that the standoff elements provide for fastening and fixation of the circuit board sections with respect to one another.

The flexible connection section may be formed by a so-called flex-band, such flex-band providing for mechanical interconnection as well as for electrical conduction paths in between the associated neighboring circuit board sections.

The circuit board sections generally may be fabricated from a conventional, substantially rigid circuit board material such as FR4, a conducting-path structure being formed on each circuit board section for providing for a desired electrical function.

In another aspect, a circuit board pre-assembly usable for fabricating a circuit board arrangement comprises: a circuit board panel forming at least one circuit board section, a remaining panel section, and at least one support portion connecting the at least one circuit board section to the remaining panel section; at least one shield element placed on the circuit board panel; and at least one functional component placed on the at least one circuit board section; wherein a shield section of the at least one shield element is arranged in between an edge of the circuit board section and a portion of the functional component to shield said portion of the functional component from interaction with an excise device when separating the at least one circuit board section from the remaining panel section at the edge of the circuit board section.

The circuit board pre-assembly represents an intermediate product which is obtained during a method of fabricating a circuit board arrangement of the kind described above. Herein, the advantages and advantageous embodiments for the method equally apply also to the pre-assembly, such that it shall be referred to the above in this respect.

In one embodiment, the at least one shield element placed on the circuit board panel such that the shield section of the at least one shield element is arranged on the at least one support portion, and the shield section of the at least one shield element is arranged in between the at least one support portion and a portion of the functional component to shield said portion of the functional component from interaction with an excise device when separating the at least one circuit board section from the remaining panel section at the at least one support portion.

In one embodiment, the shield element is made from a metal material or a ceramics material. Generally, the shield element may form a sacrificial element which may interact with an excise device and may prevent e.g. laser penetration towards a functional component. The shield element may for example be made from a metal material such as nickel, copper, steel, or another metal. In another embodiment, the shield element may be made from a ceramic material, such as a metal-plated ceramics material. In yet another embodiment, the shield element may be formed from a compound material, such as a combination of a metal and a polymer material, for example having a polymer core plated with metal. The shield element generally is thick enough to prevent penetration, e.g. laser penetration, by the excise device towards the functional component to be shielded. The shield element may be damaged during the separating step but prevents damage to the functional component.

In one embodiment, the shield element comprises a fastening section fastened to the remaining panel section, for example using a soldering technique, such as SMT soldering. As the shield element is fastened to the remaining panel section, the shield element may be discarded after separating the at least one circuit board section (with an arrangement of functional components arranged thereon) together with the remaining panel section. In other words, the shield element is fastened to the remaining panel section only, and not the circuit board sections to be excised. Consequently, the shield element is removed from the circuit board section by excision together with the remaining panel section.

In yet another aspect an implantable medical device comprises a circuit board arrangement fabricated using a method as described above. An implantable medical device in this context is configured for implantation into a patient, in particular into the heart of a patient. An implantable medical device in this context may for example be a pacemaker device, such as a leadless pacemaker, for providing for a pacing action in a patient's heart, or a defibrillator device, such as an implantable cardioverter-defibrillator (ICD), for providing for a defibrillation, or a monitoring device having a sensing function for monitoring, for example, cardiac activity of a patient. The circuit board arrangement is encapsulated within the housing and may carry one or multiple electric and/or electronic components, for example processing circuitry and communication circuitry, such as a communication coil or the like.

In one embodiment, the housing comprises a cup-shaped or can portion and a lid or flange portion, wherein the lid or flange portion is included in a feedthrough assembly comprising an electrical conductor extending through an insulator, and the flange or lid portion of the housing, wherein the insulator is arranged in a through opening in the lid or flange portion, and wherein the electrical conductor is electrically connected to the circuit board arrangement, wherein particularly the electrical conductor is joined with the circuit board arrangement, particularly by soldering. In one embodiment, the lid or flange portion and the cup-shaped or can portion of the housing have essentially the same diameter.

### BRIEF DESCRIPTION OF THE DRAWINGS

The various features and advantages of the present invention may be more readily understood with reference to the following detailed description and the embodiments shown in the drawings. Herein,

FIG. 1 shows a schematic illustration of an implantable intra-cardiac system;

FIG. 2 shows a schematic illustration of another implantable intra-cardiac system;

FIG. 3 shows a schematic illustration of a folded circuit board arrangement;

FIG. 4 shows a view of a functional component on a circuit board section prior to separating the circuit board section from a remaining panel section during a fabrication of a circuit board arrangement;

FIG. 5 shows a schematic top view of the arrangement of FIG. 4; and

FIG. 6 shows a schematic view along a cut line I-I as indicated in FIG. 5.

### DETAILED DESCRIPTION

Subsequently, embodiments of the invention shall be described in detail with reference to the drawings. In the drawings, like reference numerals shall designate functionally similar structural elements, if appropriate.

It is to be noted that the embodiments are not limiting for the invention, but merely represent illustrative examples.

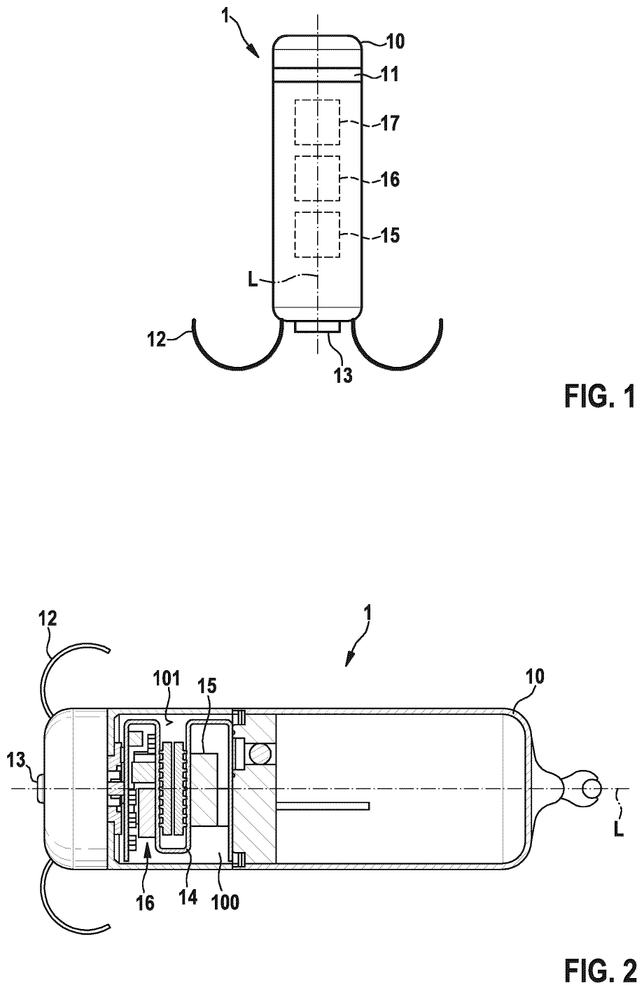

FIG. 1 shows a schematic illustration of an implantable medical device 1 in the shape of an intra-cardiac pacing system (also denoted herein as implantable leadless pacemaker). The implantable medical device 1 comprises a housing 10 which encompasses an energy storage 17 (e.g. a battery), an electronic module 16, and a communication unit having a coil arrangement 15. The housing 10 may comprise titanium or may be made of titanium.

As visible from FIG. 1, the housing 10 of the implantable medical device 1 has a generally oblong, for example cylindrical shape extending along a longitudinal axis L, and comprises a cup-shaped portion and a lid or flange portion (not indicated in FIG. 2)

At a distal end of the housing 10, i.e. the lid or flange portion, a first electrode 13 (also called pacing electrode) is disposed. In a proximal region of the housing 10, a second electrode 11 (also called sensing electrode) is arranged. The second electrode 11 may be formed as a ring electrode. Particularly, the first electrode 13 may be electrically connected and joined, particularly welded, to an electrical conductor of a feedthrough assembly 19, wherein the electrical conductor extends through an electrical insulator, the electrical insulator being arranged within an opening in the lid or flange portion and made from, e.g. a ceramic or a glass (not shown). Alternatively, the first electrode 13 may be integrally formed with the electrical conductor of the feedthrough assembly 19. On the housing side of the feedthrough assembly, the first electrode 13 or the electrical conductor, respectively, is electrically connected to the electronic module.

The implantable medical device 1 may be fixed to cardiac tissue by a fixation device 12. The fixation arrangement 12 may be formed by tines comprising Nitinol or being made of Nitinol. In one embodiment, four tines made of Nitinol may be formed at the distal end of the housing 10.

The energy storage 17 may be configured to provide electrical energy to the components of the implantable medical device 1, in particular to the electronic module 16, the coil arrangement 15, and the electrode arrangement of the first electrode 13 and the second electrode 11.

The electronic module 16 may be configured to perform the functions of a pacemaker, including sensing cardiac events and providing pacing pulses. The electronic module 16 may comprise a processor and memory.

The coil arrangement 15 may be configured for communication with an external device (e.g. a programmer wand). The coil arrangement 15 may be configured to inductively couple to an external communication coil for providing for a communication, as shall be explained further below.

In an implanted state, the implantable medical device 1, at its distal end, is placed on tissue, for example cardiac tissue of a patient's heart, such that the tines of the fixation device 12 engage with the tissue and the electrode 13 comes to rest on tissue such that it electrically contacts with the tissue. By means of the electrode arrangement formed by the electrodes 11 and 13, hence, electrical energy may be injected into the tissue for providing a stimulation, for example a pacing action or a defibrillation.

Referring now to FIG. 2, an implantable medical device 1 in the shape of a leadless pacemaker comprises a housing 10, at the distal end of which a fixation device 12 having tines for fixing the device to cardiac tissue is arranged and an electrode 13 is disposed. The implantable medical device 1 may further comprise some or all components as described above in the context of FIG. 1, in particular an energy storage 17 and an electronic module 16.

Similarly to the embodiment of FIG. 1, in the embodiment of FIG. 2 the implantable medical device 1 has an oblong shape, the housing 10 of the implantable medical device 1 extending along a longitudinal axis L. The implantable medical device 1 may for example have the shape of a cylindrical capsule, the housing 10 having a length as measured along the longitudinal axis L substantially exceeding the diameter of the housing 10 as measured in a plane perpendicular to the longitudinal axis L.

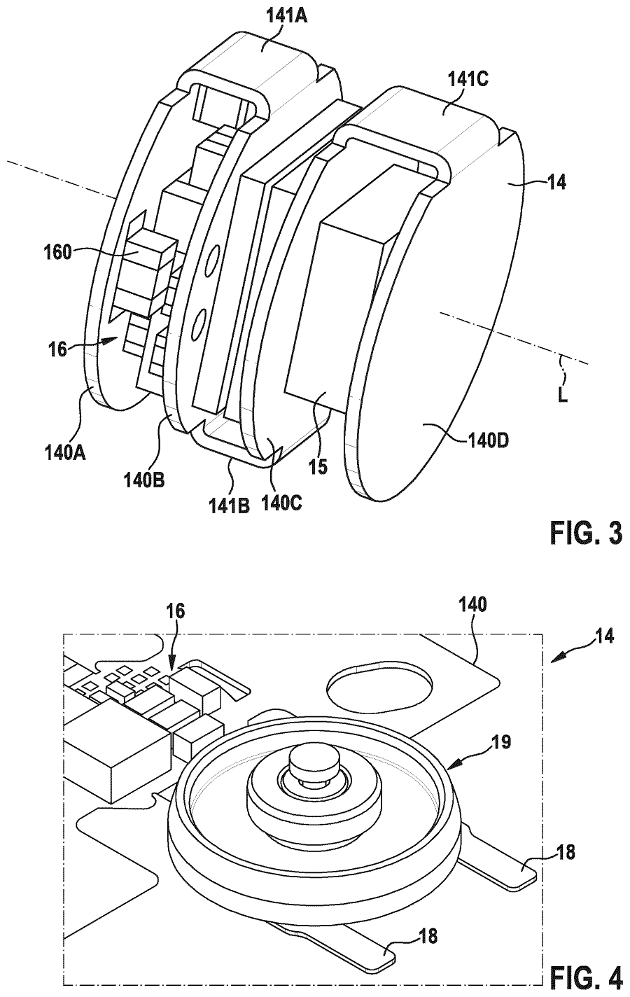

In the embodiment of FIG. 2, the implantable medical device 1 comprises a circuit board arrangement 14 comprising, in the shown embodiment, a flex-circuit printed circuit board (PCB) which in an operative state is folded into a zigzag (“accordion”) shape, as illustrated in another view in FIG. 3. The circuit board arrangement 14 comprises multiple circuit board sections 140A-140D which extend along parallel planes perpendicular to the longitudinal axis L and hence are offset with respect to each other along the longitudinal axis L. Neighboring circuit board sections 140A-140D herein are connected to each other by flexible connection sections 141A-141C such that an interlinked circuit board arrangement 14 is formed carrying electrical and electronic components of the implantable medical device 1.

Within the circuit board arrangement 14, the zig-zag shape is formed in that the circuit board sections 140A-140D are connected to each other by means of the connection sections 141A-141C in an alternating fashion at diametrically opposite sides with respect to the longitudinal axis L. In particular, a first circuit board section 140A carrying components 160 of an electronic module 16 is connected to a neighboring, second circuit board section 140B by means of a connection section 141A on a first side of the longitudinal axis L, as is visible in FIG. 3. The circuit board section 140B is connected to a neighboring, third circuit board section 140C by means of a connection section 141B, the connection section 141B being formed at a side diametrically opposite, with respect to the longitudinal axis L, to the connection section 141A. The circuit board section 140C in turn by means of a connection section 141C is connected to another, fourth circuit board section 140D, the connection section 141C again being located at a side of the longitudinal axis L diametrically opposite to the connection section 141B, as visible from FIG. 3.

The connection sections 141A-141C may be formed by so-called flex-bands mechanically interconnecting the circuit board sections 140A-140D. Conduction paths herein may be formed on the connection sections 141A-141C such that via the connection sections 141A-141C also an electrical interconnection in between the circuit board sections 140A-140D is established.

The circuit board sections 140A-140D each have a substantially circular shape, when viewed in an associated plane perpendicular to the longitudinal axis L of the implantable medical device 1. The circuit board arrangement 14 herein is received within a chamber 100 formed by the housing 10 and confined by an inner, cylindrical wall 101 surrounding the chamber 100. The shape of each circuit board section 140A-140D substantially conforms to the circular cross-sectional shape of the chamber 100, such that the circuit board arrangement 14 is received within the housing 10 in a space-efficient manner.

Because multiple circuit board sections 140A-140D are stacked and displaced with respect to each other along the longitudinal axis L, electrical and electronic components may be received within the housing 10 in a space-efficient, stacked manner, allowing to design a compact implantable medical device 1 having reduced space requirements and an increased packing density.

Electronic components 160 received on the circuit board section 140A may for example comprise a processor and a memory, for example in the shape of integrated circuits (ICs). In addition, a coil arrangement 15 may be arranged on the circuit board section 140C, the coil arrangement 15 being mechanically connected and electrically contacted to the circuit board section 140C. The coil arrangement 15 herein, as visible from FIGS. 2 and 3, may be received in between the two neighboring circuit board sections 140C, 140D.

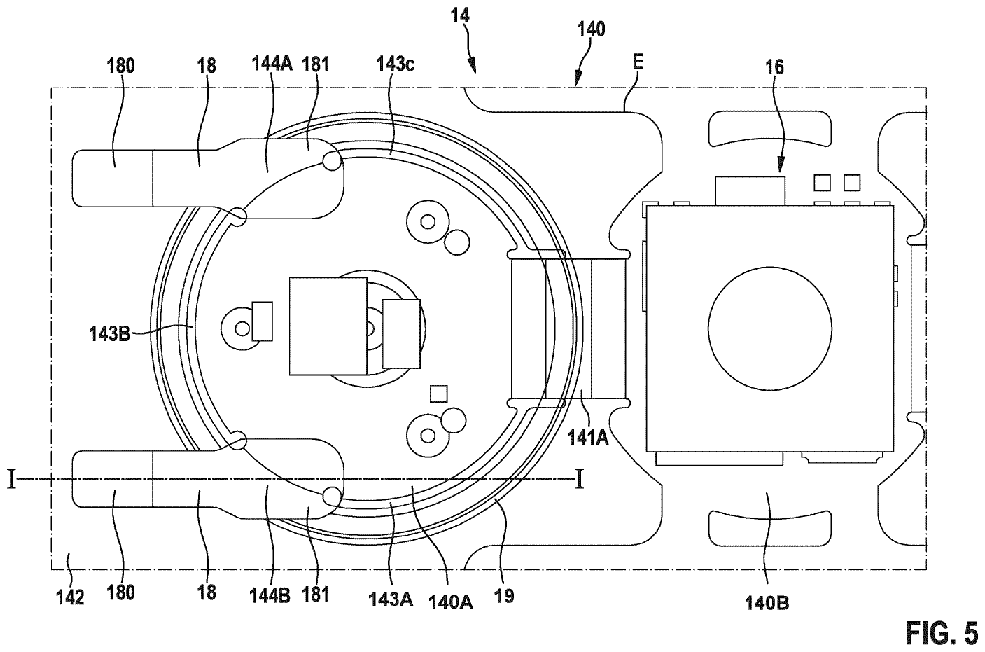

For fabricating a circuit board arrangement 14 as shown in an embodiment in FIGS. 2 and 3, circuit board sections 140A-140D are for example formed from a circuit board panel 140, as in an example shown in FIGS. 4 and 5. The circuit board panel 140 herein at the outset of fabrication is provided as a continuous, coherent panel, in which circuit board sections 140A-140D are connected to each other and are held in place by a circuit board skeleton in the shape of a remaining panel section 142. After placing functional components 15, 16 (such as a coil arrangement or components of an electronic circuitry) on the circuit board sections 140A-140D the circuit board sections 140A-140D are separated from the remaining panel section 142 in order to e.g. fold the circuit board sections 140A-140D to form the circuit board arrangement 14 as shown in FIGS. 2 and 3.

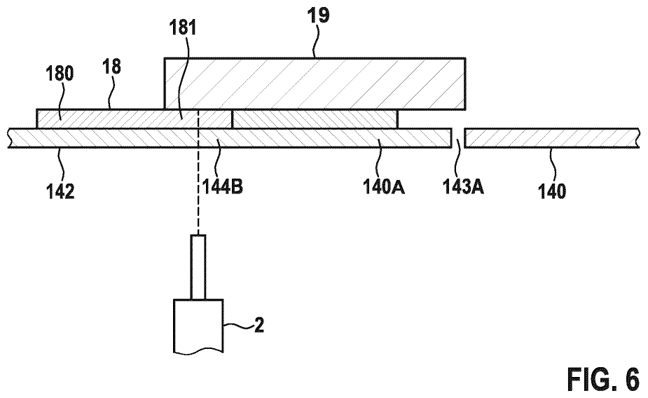

For separating the circuit board sections 140A-140D from the remaining panel section 142 within a separating step, an excise device 2 e.g. in the shape of a laser excise device is used, as it is schematically indicated in FIG. 6. The excise device 2 in particular is used to cut the circuit board panel 140 along an excise line E, as it is indicated in FIG. 5, such that the circuit board sections 140A-140D are freed from the remaining panel section 142 and may subsequently be folded in order to form the circuit board arrangement 14.

Within a fabrication process, the circuit board panel 140 initially is provided as a continuous, cohesive flat panel forming circuit board sections 140A-140D to be made into one or multiple circuit board arrangements 14. The circuit board sections 140A-140D are herein connected to a remaining panel section 142, which serves as a skeleton in order to hold the circuit board sections 140A-140D in place with respect to one another. The circuit board panel 140 extends flatly along a plane of extension, corresponding to the plane of the drawing of FIG. 5, allowing for easy handling and processing in subsequent fabrication steps.

For the fabrication, functional components such as a coil arrangement 15 and components 160 of an electronic circuitry 16 are placed on the circuit board sections 140A-140D. The functional components 15, 160 herein in particular may be Surface Mount Devices (SMD), which are placed on solder pads of the circuit board sections 140A-140D and are subsequently joined to the circuit board sections 140A-140D in a soldering process step, in particular using a reflow soldering technique in which all components 15, 16 are joined to the circuit board panel 140 simultaneously. Subsequently, the circuit board panel 140, particularly circuit board section 140A, is electrically connected and joined with a feedthrough assembly 19, particularly by soldering, wherein the feedthrough assembly 19 comprising a flange portion made of, for example titanium, an electrical insulator made from, e.g. a ceramic, a glass or glass solder, or a plastic, and an electrical conductor extending through the electrical insulator. The first electrode 13 may be joined with the electrical conductor, e.g. by welding, or the first electrode 13 and the electrical conductor may be integrally formed in one piece. Then the circuit board sections 140A-140D are separated from the remaining panel section 142 using the excise device 2 along the excise line E, as indicated in FIG. 5.

As apparent from FIGS. 4 and 5, a functional component such as feedthrough assembly 19 may be comparatively large in comparison to the dimensions of an associated circuit board section 140A, the circuit board section 140A having a rather small size in order to be fitted into a chamber 100 of a housing 10 e.g. of an implantable medical device 1, as it is shown in FIG. 2. The functional component 19 hence may reach across or extend beyond the excise line E along which the circuit board section 140A, during the separating step, shall be separated from the remaining panel section 142.

This may bring about a potential risk of damaging the feedthrough assembly 19, or other overhanging functional components during the separating step, as the excise device 2, for example in the shape of a laser excise device, may impinge on the feedthrough assembly 19 and hence may potentially damage the feedthrough assembly 19 or other overhanging functional components.

In order to reduce a risk of damaging the feedthrough assembly 19 or other overhanging functional components during the separating step, pre-rout openings 143A, 143B, 143C are formed in the circuit board panel 140 prior to placing the components 15, 16, and 19 on the circuit board panel 140 during the fabrication. Hence, portions of the excise line E are already cut prior to placing the components 15, 16, and 19 on the circuit board panel 140, such that minimal cutting along portions of the excise line E with overhanging components is required during the separating step.

However, in the example of FIG. 5, at support portions 144A, 144B the circuit board section 140A carrying the feedthrough assembly 19 is connected to the remaining panel section 142 in order to provide for support of the circuit board section 140A with respect to the remaining panel section 142. Such support is advantageous during fabrication because e.g. during a solder print step, in which solder paste is printed onto the circuit board panel 140 to form solder pads, the circuit board section 140B preferably should not deflect, which would lead to poor solder paste print performance.

During the separating step, hence, the circuit board section 140B needs to be excised at the support portions 144A, 144B using the excise device 2. Herein, to provide for protection of the feedthrough assembly 19 in the region of the support portions 144A, 144B, shield elements 18 are placed on the circuit board panel 140 in order to shield the feedthrough assembly 19 from the excise device 2 during the separating step.

As visible from FIG. 5, the shield elements 18 are fastened, at fastening sections 180, to the remaining panel section 142, for example by solder connections. The shield elements 18 herein are placed on the circuit board panel 140 such that shield sections 181 are arranged over the support portions 144A, 144B to cover the support portions 144A, 144B in a region in which the support portions 144A, 144B are excised in a subsequent separating step.

During fabrication, the shield elements 18, in one embodiment, are placed on the circuit board panel 140 prior to feedthrough assembly 19. The shield elements 18 herein are placed immediately on the circuit board panel 140, such that the fastening sections 180 for example come to lie on solder pads on the remaining panel section 142 and the shield sections 181 reach across the support portions 144A, 144B, as it is visible from FIG. 5. Subsequently, the flange portion 19 is placed on the circuit board section 140A, such that the shield sections 181 are arranged in between the circuit board panel 140 and the flange portion 19 in the region of the support portions 144A, 144B, as it is schematically indicated in FIG. 6.

The shield elements 18 and the feedthrough assembly 19 are arranged on a common side of the circuit board panel 140, as visible from FIG. 6. If now, during the separating step, the excise device 2 is used on an opposite, second side of the circuit board panel 140 in that e.g. a laser beam of a laser excise device 2 is directed towards the circuit board panel 140 at the support portions 144A, 144B, the excise device 2 cuts the support portions 144A, 144B, but an interaction of the excise device 2 with the feedthrough assembly 19 is prevented by the shield elements 18, as visible from FIG. 6.

The shield elements 18, by means of their shield sections 181, hence provide for shielding in that they prevent impingement or penetration e.g. of a laser beam of the excise device 2 towards the feedthrough assembly 19. The laser beam hence cuts the circuit board panel 140 at the support portions 144A, 144B, but may not penetrate through the shield elements 18 and hence does not act on the feedthrough assembly 19, such that damaging of the feedthrough assembly 19 is effectively prevented.

The shield elements 18 may for example be made from a metal material, such as a nickel, copper or steel, or a ceramic material, e.g. a ceramic material plated with metal. The shield elements 18 are thick enough such that e.g. a laser beam of the excise device 2 may not penetrate through the shield elements 18 during the separating step. The shield elements 18 provide for sacrificial components which may be discarded after the separating step.

By means of the fastening sections 181 the shield elements 18 are connected to the remaining panel section 142. After the separating of the circuit board sections 140A-140D from the circuit board panel 140, the remaining panel section 142 is removed and may be discarded together with the shield elements 18 arranged thereon. Hence, the shield element 18 is particularly fastened to the remaining panel section 142 only, i.e. and not fastened to the any one of the circuit board sections 140A-140D.

The shield elements 18, together with the functional component 15, 16, may be automatically placed on the circuit board panel 140 using an automated placement machine during the fabrication of the circuit board arrangement 14. Hence, cost and cycle time are not substantially increased by providing the shield elements 18.

The use of shield elements 18 as described herein may help to improve soldering yields and solder joint quality by improving rigidity of a printed circuit board panel 140 (by increasing a support of circuit board sections 140A-140D with respect to a remaining panel section 142). In addition, pre-rout features may be limited or fully eliminated, hence helping to reduce manufacturing costs.

A circuit board arrangement 14 as described herein may provide for an electronic circuit assembly, in particular within an implantable medical device 1. It shall be noted herein, however, that a circuit board arrangement 14 of the kind described in this text may also be usable in other electrical and electronic devices, the invention hence not being limited to implantable medical devices 1.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE NUMERALS

1 Implantable medical device (pacemaker device)
10 Housing
100 Chamber
101 Inner wall
11 Electrode
12 Fixation device
13 Electrode
14 Circuit board arrangement
140 Circuit board panel
140A-140D Circuit board section
141A-141C Connection sections (flex-bands)
142 Remaining panel section (panel skeleton)
143A-143C Pre-rout openings
144A, 144B Support portions
15 Coil arrangement
16 Electronic module
160 Electronic components
17 Energy storage
18 Shield element
19 Feedthrough assembly
180 Fastening section
181 Shield section
2 Excise device (laser)
E Excise line
L Longitudinal axis

1. A method for fabricating a circuit board arrangement, comprising:
providing a circuit board panel forming at least one circuit board section, a remaining panel section and at least one support portion connecting the at least one circuit board section to the remaining panel section;
placing at least one functional component on the at least one circuit board section; and
separating the at least one circuit board section from the remaining panel section at the at least one support portion using an excise device,
wherein
a portion of the at least one functional component extends beyond an edge of the at least one circuit board section, and
at least one shield element is arranged on the circuit board panel such a shield section of the at least one shield element is at least partly arranged between the at least one functional component and the edge of the circuit board section.

2. The method according to claim 1, wherein said step of placing the at least one shield element on the circuit board panel is carried out prior to said step of placing the at least one functional component on the at least one circuit board section.

3. The method according to claim 1, wherein the at least one shield element comprises a fastening section for fastening the at least one shield element to the circuit board panel, wherein said step of placing the at least one shield element on the circuit board panel includes placing the fastening section on the remaining panel section.

4. The method according to claim 1, including connecting, after said step of placing the at least one shield element on the circuit board panel and said step of placing the at least one functional component on the at least one circuit board section, said at least one shield element and said at least one functional component to the circuit board panel using a soldering technique.

5. The method according to claim 1, wherein the functional component is a feedthrough assembly of a medical device.

6. The method according to claim 1, wherein said step of placing the at least one shield element on the circuit board panel includes placing the at least one shield element on a first side of the circuit board panel, and said step of placing the at least one functional component on the at least one circuit board section includes placing the at least one functional component on said first side of the circuit board panel.

7. The method according to claim 6, wherein said step of separating includes excising the at least one support section using the excise device on a second side of the circuit board panel opposite the first side.

8. The method according to claim 1, wherein the excise device is a laser excise device.

9. The method according to claim 1, wherein the circuit board panel comprises at least one pre-rout opening partially separating the at least one circuit board section from the remaining panel section.

10. The method according to claim 9, wherein said step of placing the at least one functional component on the at least one circuit board section includes placing the at least one functional component on the at least one circuit board section such that a portion of the functional component reaches across said at least one pre-rout opening.

11. The method according to claim 1, wherein the circuit board panel forms at least two circuit board sections and a flexible connection section connecting the at least two circuit board sections to each other.

12. A circuit board pre-assembly usable for fabricating a circuit board arrangement, comprising:
a circuit board panel forming at least one circuit board section, a remaining panel section and at least one support portion connecting the at least one circuit board section to the remaining panel section;
at least one shield element placed on the circuit board panel; and
at least one functional component placed on the at least one circuit board section;
wherein a shield section of the at least one shield element is arranged in between an edge of the at least one circuit board section and a portion of the functional component to shield said portion of the functional component from interaction with an excise device when separating the at least one circuit board section from the remaining panel section at the edge of the at least one circuit board section.

13. The circuit board pre-assembly according to claim 12, wherein said at least one shield element is made from a metal material or a ceramic material.

14. The circuit board pre-assembly according to claim 12, wherein said at least one shield element comprises a fastening section fastened to the remaining panel section.

15. An implantable medical device comprising a circuit board arrangement fabricated using the method according to claim 1.
